(19) United States
(12) Patent Application Publication
(10) Pub. No.: US 2025/0041169 A1
(43) Pub. Date: Feb. 6, 2025

(54) POWDERED COSMETIC COMPOSITION

(71) Applicant: Conopco, Inc., d/b/a UNILEVER, Englewood Cliffs, NJ (US)

(72) Inventors: Hasiba BEKTO, Bristol, CT (US); Jenna Christine DOUTHIT, Derby, CT (US); Kayla Marie KEMLER, Milford, CT (US); Anjing LOU, Seymour, CT (US)

(73) Assignee: Conopco, Inc., d/b/a UNILEVER, Englewood Cliffs, NJ (US)

(21) Appl. No.: 18/717,446

(22) PCT Filed: Nov. 29, 2022
(86) PCT No.: PCT/EP2022/083660

§ 371 Date: Jun. 7, 2024

(30) Foreign Application Priority Data
Dec. 21, 2021 (EP) 21216563.3

(51) Int. Cl. A61K 8/02; A61K 8/34; A61K 8/36; A61K 8/41; A61K 8/73; A61Q 5/12; A61Q 19/10

(52) U.S. Cl. CPC A61K 8/022; A61K 8/342; A61K 8/361; A61K 8/416; A61K 8/731; A61K 8/732; A61Q 5/12; A61Q 19/10; A61K 2800/412

(57) ABSTRACT

Disclosed herein are personal care compositions. More particularly, disclosed herein is a rapidly hydratable, powdered cosmetic compositions comprising a quaternary ammonium compound mixture comprising a quaternary ammonium compound mixture comprising a quaternary ammonium compound, fatty acid, fatty alcohol, or combination thereof, and hydration enhancer; and a stabilizing carrier. Such a powdered cosmetic composition unexpectedly remains free-flowing under high humidity and/or high temperature conditions, is convenient to evacuate from packaging.

### FIELD OF THE INVENTION

Disclosed herein are personal care compositions. More particularly, disclosed herein are rapidly hydratable, powdered cosmetic compositions comprising a quaternary ammonium compound mixture comprising a quaternary ammonium compound, fatty acid, fatty alcohol, or combination thereof, and a hydration enhancer; and a stabilizing carrier. Such a powdered cosmetic composition unexpectedly remains free-flowing under high humidity and/or high temperature conditions, and is convenient to evacuate from packaging.

### BACKGROUND OF THE INVENTION

It is often publicized that the world's oceans will soon have more plastic than fish. Given environmental concerns and the desire for consumers and conscious companies to do more for the planet, there is a strong desire to use less plastic when selling products, including consumer products. In view of this, efforts have been made to sell product in concentrate form, and therefore, to ship product that comprises less water. The difficulty with concentrates is that consumers often find concentrates to be difficult to dispense, as many are not humidity- and/or heat-proof, thereby resulting in undesirable agglomeration. This is of particular importance when the concentrate is intended to be used in a steamy environment such as a residential bathroom. Other complaints include that the concentrate does not hydrate well or does not produce a homogenous end use product.

It is of increasing interest to develop a powdered cosmetic composition that is easy to carry and hydrate, and that results in a consumer-desirable homogeneous end use product with limited shear and water. It is also of increasing interest to have a powdered cosmetic composition that can be easily used to prepare products for single-use applications. This invention, therefore, is directed to a powdered cosmetic composition that comprises a powdered quaternary ammonium compound mixture of quaternary ammonium compound mixture comprising a quaternary ammonium compound, fatty acid, fatty alcohol, or combination thereof, and a hydration enhancer; and a stabilizing carrier. Such a powdered cosmetic composition unexpectedly does not aggregate, is convenient to evacuate from packaging and is easy to hydrate for a single use application.

Therefore, the present inventors have recognized a need to develop a powdered cosmetic composition that does not aggregate, is convenient to evacuate from packaging and is easy to hydrate for a single use application. Thus, disclosed herein is a powdered cosmetic composition having a powdered quaternary ammonium compound mixture of quaternary ammonium compound mixture comprising a quaternary ammonium compound, fatty acid, fatty alcohol, or combination thereof, and a hydration enhancer; and a stabilizing carrier.

### Additional Information

Efforts have been disclosed for making powdered cosmetic compositions.

U.S. Pat. No. 5,472,688 discloses a process for obtaining a hair conditioner which imparts gloss, softness, and elasticity to hair comprising ascorbic acid and cupric and/or cupric salts. In one version, the conditioner is in powder form.

U.S. Pat. No. 7,670,998 describes a cosmetic product for conditioning hair having cocoa butter, cetearyl alcohol, sodium lauryl sulfate, glyceryl stearate, and PEG-100 stearate to form a solid, and further comprising at least one hair conditioning ingredient such as lanolin and cetrimonium bromide.

U.S. Patent Application Publication No. US2008138308 discloses a base composition comprising non-ionic surfactant and cationic surfactant wherein the base composition is used to prepare a hair conditioner by mixing with water.

U.S. Patent Application Publication No. US 20030103930 relates to cosmetic compositions comprising a quaternary ammonium compound having at least one group selected from the groups consisting of an ester group, an amino group, and mixtures thereof; and an anhydrous carrier.

None of the additional information describes a powdered cosmetic composition as described and claimed in the present application.

### SUMMARY OF THE INVENTION

The present inventors have found a powdered cosmetic composition having a powdered quaternary ammonium compound mixture of quaternary ammonium compound mixture comprising a quaternary ammonium compound, fatty acid, fatty alcohol, or combination thereof, and a hydration enhancer; and a stabilizing carrier, wherein the powdered cosmetic composition does not aggregate, is convenient to evacuate from packaging and is easy to hydrate for a single use application.

Accordingly, in a first aspect, the inventive composition relates to a powdered cosmetic composition comprising
a) a quaternary ammonium compound mixture comprising a quaternary ammonium compound; a C₁₄₋₂₂ fatty acid, fatty alcohol, or combination thereof; and a hydration enhancer; and
b) stabilizing carrier,
   wherein the quaternary mixture has a hardness from 250 to 600 positive force (g) and an average particle size from 20 to 250 microns.

In a second aspect, the inventive composition disclosed is a method of conditioning with the powdered cosmetic composition of the first aspect by hydrating the composition with shear to produce an end use cosmetic composition and contacting skin, hair, nails or an inanimate object with the end use cosmetic composition.

In a third aspect, disclosed herein is directed to the use of a composition comprising
a) a quaternary ammonium compound mixture comprising a quaternary ammonium compound; a C₁₄₋₂₂ fatty acid, fatty alcohol, or combination thereof; and a hydration enhancer; and
b) stabilizing carrier,
   to condition skin, hair, nails or an inanimate object.

All other aspects of the present cosmetic compositions will become more readily apparent upon considering the detailed description and examples which follow.

As used herein, “quaternary ammonium compound mixture” or “quaternary mixture” refers to a blend comprising an ammonium quaternary ammonium compound, C₁₀₋₂₂ fatty alcohol, and a hydration enhancer. “Hydration enhancer” as used herein means a water-soluble or water-dispersible material. As used herein, the terms “dry,” “solid” or “anhydrous” are to be used interchangeably and means essentially free of water (i.e., the presence of 0 to 5% water, preferably, 0.01 to 3%, and, optimally, 0.05 to 1% water by weight of the specified ingredient). Water of hydration is not considered to be water for purposes of the anhydrous definition, but it is preferred to minimize, preferably to eliminate any water of hydration. “Stabilizing carrier” means a natural material such as clay or talc and/or a carbohydrate or starchy material that can stabilize and retain moisture.

The term “skin” as used herein includes the skin on the face, neck, chest, back, arms, axilla, buttocks, hands, legs and scalp. Skin benefit agent, as used herein, is meant to include a component that improves a facial or body characteristic after topical application like a skin characteristic and/or benefits the same wherein the skin benefit agent can be, and preferably is, a skin benefit agent in a cream, pump or aerosol spray, serum, lotion, balm, deodorant, gel, or non- or low-foaming wash composition. The powdered cosmetic composition after hydration (i.e., combining with water) is herein referred to as an end use (cosmetic) composition that is interchangeable with “hydrated product.” Therefore, the powdered cosmetic composition itself is a precursor to an end use composition. The end use cosmetic composition includes conditioners, lotions, creams, balms, deodorant, shaving creams, masks, and non-foaming cleansers. Powder flow, as used herein, means easy to shake out of a shaker, where powder clumping and static cohesion are not visually observed. Similarly, as used herein, the term “free-flowing” is meant to describe a powder that moves in a continuous steady flow. Conditions of high humidity and high temperatures as used herein refers to 75% relative humidity at 40° C. Substantially free of, as used herein, is intended to mean comprising less than 5% by weight, preferably, less than 2% by weight, more preferably, less than 0.5% by weight of the composition.

Unless explicitly stated otherwise, all ranges described herein are meant to include all ranges subsumed therein. The term comprises is meant to encompass the terms consisting essentially of and consisting of. For the avoidance of doubt, a composition comprising a quaternary ammonium compound mixture and stabilizing carrier is meant to include a composition consisting essentially of the same and a composition consisting of the same. As to the percentages used herein, the same are meant to be by weight of ingredient, (e.g., not including any water it may be supplied with) unless noted otherwise. Except in the operating and comparative examples, or where otherwise explicitly indicated, all numbers used in this description indicating amounts, or ratios of materials and/or use thereof are to be understood as modified by the word “about.”

### DETAILED DESCRIPTION OF THE INVENTION

Powdered cosmetic compositions disclosed herein comprise a quaternary ammonium compound mixture comprising a quaternary ammonium compound; a C₁₄₋₂₂ fatty acid, fatty alcohol, or combination thereof; and a hydration enhancer further with a stabilizing carrier. In a preferred aspect, the disclosed powdered cosmetic composition is anhydrous.

Desirable quaternary ammonium compounds are preferably solid at 25° C. Quaternary ammonium compounds desirable for use in the disclosed powdered cosmetic compositions possess the chemical structure:

[R—N(CH₃)₃]⁺X⁻,

wherein R is a C₁₄₋₂₂ alkyl group, preferably, a C₁₆₋₂₂ alkyl group,
and wherein X is a halogen, phosphate, sulfate, or carbonate anion.

In a preferred aspect, X is a chloride anion.

Desirable quaternary ammonium compounds for use in the compositions disclosed herein include those that are characterized by a single fatty chain attached a cationic headgroup such as behentrimonium chloride, stearyl trimethyl ammonium chloride, and behentrimonium methosulfate. Behentrimonium chloride is commercially available as Genamin® BTLF from Clariant and as AMBH2D6EDE6A from Ambeed, Inc. Stearyl trimethyl ammonium chloride is made commercially available as STAC 808KC from the supplier KCI Limited. Behentrimonium methosulfate is commercially available as Genamin® BTMS from Clariant. The quaternary ammonium compounds may be included in the powdered cosmetic compositions in amounts of 10 to 55%, preferably, 15 to 50%, and, more preferably, 20 to 45% by weight of the quaternary mixture.

Quaternary ammonium compound mixtures of the disclosed powdered cosmetic compositions also comprise C₁₄₋₂₂ fatty acid, fatty alcohol, or combination thereof. In a preferred aspect, the fatty acid and/or fatty alcohol is of chain length C₁₆₋₂₂, more preferably, C₁₈₋₂₂. Desirable fatty acids and fatty alcohols have a melting point greater than 40° C., preferably, greater than 45° C., and, more preferably, greater than 49° C. The fatty acid, fatty alcohol, or combinations thereof may be included in the powdered cosmetic compositions in amounts of 30 to 65%, preferably, 35 to 60%, and, more preferably, 40 to 55% by weight of the quaternary mixture.

The quaternary mixture of the inventive compositions disclosed is characterized by a weight ratio of quaternary ammonium compound to fatty acid and/or fatty alcohol of 20:80 to 80:20, preferably, 25:75 to 75:25, more preferably, 30:70 to 70:30. In a preferred aspect, the quaternary ammonium compound mixture comprises a 40:60 to 60:40 weight ratio of quaternary ammonium compound to fatty acid/alcohol.

Desirable hydration enhancers comprise tapioca starch, rice starch, corn starch, citrus fibers, microcrystalline cellulose, hydroxypropyl starch phosphate, and combinations thereof. Without intending to be bound or limited by theory, it is believed that hydration enhancers help to draw water into the composition to allow for full and faster hydration. Quaternary ammonium compound mixtures as in the disclosed compositions comprise at most 50%, preferably, 2.5 to 40%, more preferably, 5 to 20% hydration enhancers by weight of the composition.

The quaternary mixture is evaluated using hardness as a discerning parameter to determine whether or not the mixture itself may be reduced efficiently to desirable particle sizes disclosed herein through milling or spray-cooling techniques known to a skilled artisan. Hardness may be measured with art recognized instrumentation, such as a TA.XT Plus Texture Analyzer as manufactured by Stable Micro Systems, using a 30° conical probe for such measurements. Hardness measurements disclosed herein were measured with the test mode set as Compression and a target mode set as Distance. Detailed conditions include a pre-test speed of 10 mm/s, a test speed of 1 mm/s, a distance of 1 mm, and a trigger force of 0.005 kilogram (kg). Desirable hardness of the quaternary ammonium compound mixture is found within the range of 250 to 600 positive force (g), preferably, 300 to 550 g.

The quaternary mixture used in the powdered cosmetic composition of this invention typically makes up a total of 2 to 70%, preferably, 5 to 65%, and, more preferably, 7 to 60% by weight of the powdered cosmetic composition. In one aspect, the quaternary mixture may be included in an amount of 10 to 55% by weight of the powdered cosmetic composition. In another aspect, the quaternary mixture makes up from 15 to 45% by weight of the powdered cosmetic composition.

The disclosed powdered cosmetic composition also comprises a stabilizing carrier. Such a stabilizing carrier has been determined to unexpectedly contribute to an enhanced stability in conditions characterized by high humidity and high temperature. A representative condition of high humidity and high temperature is 75% relative humidity (RH) at 40° C. for two (2) weeks (i.e., 14 days). Without wishing to be bound by theory, it is believed that such a system helps to protect powdered materials in the powdered cosmetic composition from caking and settling under high humidity and high temperature conditions.

The stabilizing carrier used is limited only to the extent that the same is suitable for use in a topical composition. These includes include fibers, starches, clays (e.g., kaolin, bentonite) or clay minerals (e.g., talc), silicone and/or elastomer powder, and non-modified cellulose (e.g., cellulose microfibrils, cellulose nanocrystals or microcrystalline cellulose). Representative of the starches are chemically modified starches such as sodium hydroxypropyl starch phosphate and aluminum starch octenylsuccinate and non-modified starches such as corn starch, rice starch, tapioca starch, and maltodextrin. It is preferred that the stabilizing carrier comprises corn starch, tapioca starch, rice starch, microcrystalline cellulose, clay or clay mineral, silicone and/or elastomer powder, or a mixture thereof. As to stabilizing carrier used in the powdered cosmetic composition, 20 to 90%, preferably, 22 to 80%, and, more preferably, 25 to 70% by weight stabilizing carrier in the powdered cosmetic composition. In a particularly preferred aspect, 27 to 60% by weight stabilizing carrier is used in the powdered cosmetic composition.

In an aspect of the present application, the stabilizing carrier used is rice starch and/or a corn starch referred to as INCI: Zea mays (Corn) Starch like, for example, the product sold under the commercial name Farmal™ CS 3757 by Ingredion. In another aspect of the present application, a stabilizing carrier used is unmodified rice starch referred to as INCI: Oryza sativa (rice) starch that is also made commercially available from Ingredion under the Nativacare™ name. In still another aspect of the present application, a stabilizing carrier used is microcrystalline cellulose made available by Active Organics under the name Acticel® 12 name (CAS 9004-34-6).

In still another aspect of the present application, desirable stabilizing carrier include clays or clay minerals like, for example, bentonite, kaolin, or talc. In a further aspect, stabilizing carriers may also include silicone and/or elastomer powders such as that made commercially available as DOWSIL™ EP-9801 Hydro Cosmetic Powder (INCI: Dimethicone/Vinyl Dimethicone Crosspolymer (and) Silica (and) Butylene Glycol) by Dow®.

Desirable sources of cellulose microfibrils include secondary cell wall materials (e.g., wood pulp, cotton), bacterial cellulose, and primary cell wall materials. Preferably, the source of primary cell wall material is selected from parenchymal tissue from fruits, roots, bulbs, tubers, seeds, leaves and combination thereof; more preferably is selected from citrus fruit, tomato fruit, peach fruit, pumpkin fruit, kiwi fruit, apple fruit, mango fruit, sugar beet, beet root, turnip, parsnip, maize, oat, wheat, peas and combinations thereof; and even more preferably is selected from citrus fruit, tomato fruit and combinations thereof. A most preferred source of primary cell wall material is parenchymal tissue from citrus fruit. Citrus fibers, such as those made available by Herbacel® as AQ Plus can also be used as source for cellulose microfibrils. The cellulose sources can be surface modified by any of the known methods including those described in Colloidal Polymer Science, Kalia et al., “Nanofibrillated cellulose: surface modification and potential applications” (2014), Vol 292, Pages 5-31.

The stabilizing carrier can be present either as generally round or irregular shape particles. Such particles are also desirably hydrophilic. In an aspect of the inventive powdered cosmetic composition, the particles may have an average particle size from 0.5 to 350 microns, and preferably from 5 to 300 microns, and most preferably, from 20 to 250 microns. Average particle size as used herein means the volume-mean particle size that refers to the diameter of the particle in the aqueous dispersion. For polymer particles that are not spherical, the diameter of the particle is the average of the long and short axes of the particle. Particle sizes can be measured on a Beckman-Coulter LS 13 320 laser-diffraction particle size analyzer, with an art recognized sieve or any other art recognized device.

In one aspect, the powdered cosmetic compositions disclosed herein are substantially free from inorganic heat generating agents. In a preferable aspect, the powdered cosmetic compositions disclosed herein do not contain inorganic heat generating agents that are known in the relevant field to generate heat upon mixing with water. Illustrative examples include metal chlorides such as calcium chloride and magnesium chloride; metal oxides such as calcium oxide and magnesium oxide; metal carbonates such as sodium carbonate; and metal sulfates such as calcium sulfate, magnesium sulfate and zinc sulfate.

When using the powdered cosmetic composition of the present invention, powder and water, in no particular order, can placed in a mixing vessel and shaken, stirred and/or agitated with moderate shear. The resulting end use cosmetic composition can be used as desired (e.g., on hands, face, body, and/or hair). The amount of end use cosmetic composition made is determined by consumer preference. For a single use application to the hand, face, body or hair, typically from 0.25 to 12 grams, preferably, from 0.3 to 10 grams, and, most preferably, from 0.5 to 8 grams of powdered cosmetic composition is used. The amount of water used with the powdered cosmetic composition to make end use composition is also determined by consumer preference. Often, the amount/weight of water used with powdered cosmetic composition to produce end use cosmetic composition is from 1 to 12 times, preferably, from 3 to 10 times, and, most preferably, from 4 to 8 times the amount/weight of powdered cosmetic composition used.

In an embodiment, powdered cosmetic composition and water, in no particular order, are placed in the hand. Mixing and shearing with both hands results in an end use cosmetic composition generation. End use cosmetic composition for hair conditioning can be produced in the hands as well. In an embodiment of the invention, water and cleansing composition can be placed in a hand and subsequently supplied to the face or head where the application motion provides the shear to make the face or hair composition as the case may be.

Optional ingredients that may be used in the powdered cosmetic composition of the present invention include preservatives to assist against the growth of potentially harmful microorganisms when the end use cosmetic composition is made. Suitable traditional preservatives that may be used include hydantoin derivatives, propionate salts, and a variety of quaternary ammonium compounds. Often preferred preservatives are potassium sorbate, iodopropynyl butyl carbamate, phenoxyethanol, methyl paraben, wasabi-based preservatives, propyl paraben, imidazolidinyl urea, sodium dehydroacetate and benzyl alcohol. Especially preferred additives suitable to be employed in the powdered cosmetic composition of the present invention are 1,2-alkanediols like 1,2-octanediol, 1,2 hexanediol or mixtures thereof. In one aspect, the powdered cosmetic composition may comprise of a preservative system that is formaldehyde-free, paraben-free, or both. In another aspect, the powdered cosmetic composition may be free of preservative. In yet another aspect, the powdered cosmetic composition is substantially free of silicones, sulfates, or a combination thereof. In still another aspect, the powdered cosmetic composition does not contain silicone, sulfate, or both.

Traditional fragrance components like eugenol, coumarin, linalyl acetate, citronellal, iris concentrate, terpinyl acetate, terpineol, thymol, pinenes (e.g., alpha and beta pinene) and citronellol may optionally be added to the powdered cosmetic composition as well.

If employed, the traditional preservatives, vicinal diol and/or fragrance component will not make up more than 2%, and preferably, not more than 1%, and most preferably, from 0.2 to 0.85% by weight of the end use cosmetic composition of the present invention. In an embodiment of this invention from 0.2 to 0.8% by weight optional preservative, vicinal diol and/or fragrance component is used, based on total weight of the end use cosmetic composition. In an embodiment of the invention, no traditional preservative, vicinal diol and/or fragrance component (except for what may be provided in the fragrance used in the end use cosmetic composition) is used in the end use cosmetic composition since such a cosmetic composition can be made and used on demand if desired giving the consumer the option to have a preservative-free cosmetic product.

Other optional ingredients suitable for use include zinc pyrithione, octopirox, or a mixture thereof, especially when the end use cosmetic composition is a conditioner that provides antidandruff benefits. Each of these substances may range from 0.05 to 3%, preferably between 0.1 and 2% by weight of the total weight of the end use cosmetic composition.

Additional optional ingredients that may be used include sensory oils and/or exfoliants. Desirable oils include rose, lime, coconut, lavender, argan, sweet almond oil(s) or mixtures thereof. Illustrative exfoliants desirable for use include salt, sugar, apricot, walnut shell, rice, nutmeg and/or oatmeal powder(s). When used, sensory oils and exfoliants can make up from 0.1 to 2% by weight of the end use cosmetic composition, with the proviso that the total amount of fragrance and sensory oil does not exceed 2.5% by weight of the end use cosmetic composition, and preferably, not more than 2.0 percent by weight of the composition.

The cosmetic powder composition disclosed herein may include vitamins. Illustrative vitamins are Vitamin B₂, Vitamin B₃ (niacinamide), Vitamin B₆, Vitamin C, Vitamin E, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. Total amount of vitamins when present may range from 0.001 to 10%, and preferably from 0.01% to 5%, optimally from 0.1 to 3% by weight of the end use cosmetic powder composition.

Other optional additives desirable for use include resorcinols like 4-ethyl resorcinol, 4-hexyl resorcinol, 4-phenylethyl resorcinol, dimethoxytoluyl propyl resorcinol, 4-cyclopentyl resorcinol, 4-cyclohexylresorcinol, thiamidol; alpha- and/or beta-hydroxyacids; retinoic acid and its derivatives (e.g., cis and trans); retinal; retinol; retinyl esters such as retinyl acetate, retinyl palmitate, and retinyl propionate; petroselinic acid; conjugated linoleic acid; 12-hydroxystearic acid; mixtures thereof or the like. Still other optional additives like ethanol, quaternary ammonium compounds (like cetrimonium chloride, benzalkonium chloride or the like) and may also be included. Further optional additives including emulsifiers may be used in the cleaving powder composition. Conditioning agents such as polyquaternium compounds (e.g., polyquaternium-67) may also be desirable for inclusion in the inventive composition. Such additives, when used, collectively make up from 0.001 to 3%, preferably, from 0.01 to 2%, and, most preferably, from 0.1 to 1.5% by weight of the end use cosmetic composition.

Desquamation promoters may be present. Illustrative are the alpha-hydroxycarboxylic acids, beta-hydroxycarboxylic acids. The term “acid” is meant to include not only the free acid but also salts and C₁-C₃₀ alkyl or aryl esters thereof and lactones generated from removal of water to form cyclic or linear lactone structures. Representative acids are glycolic and its derivatives, lactic and malic acids. Amounts of these materials when present may range from 0.01 to 3%, and, preferably, from 0.1 to 2% by weight of the end use cosmetic composition.

A variety of herbal extracts may optionally be included in the cosmetic compositions of this invention. Illustrative extracts include those removed from green tea, yarrow, ginseng, marigold, hibiscus, Ginko biloba, chamomile, licorice, aloe vera, grape seed, citrus unshiu, willow bark, sage and rosemary. Humectants like glycerol and other polyols may also be included. Humectants and/or extracts, such as sorbitol, when used, typically make up from 0.01 to 5%, preferably, from 0.01 to 4%, and, most preferably, from 0.02 to 3% by weight of the end use composition.

Another optional additive suitable for use includes hemp oil with 2.5 to 25% by weight cannabigerol and/or cannabidiol at from 0.5 to 10 percent by weight. When used, such oil makes up from 0.0001 to 12% by weight of the composition, and preferably, from 0.01 to 5% by weight of the end-use composition, including all ranges subsumed therein.

Also optionally suitable for use include materials like chelators (e.g., EDTA), opacifiers (like TiO₂, particle size from 50 to 1200 nm, and preferably, 50 to 350 nm), kaolin, bentonite, zinc oxide, iron oxide, mica, C₈₋₂₂ fatty acid substituted saccharides, lipoic acid, retinoxytrimethylsilane (available from Clariant Corp. under the Silcare 1M-75 trademark), dehydroepiandrosterone (DHEA) or mixtures thereof. Ceramides (including Ceramide 1, Ceramide 3, Ceramide 3B and Ceramide 6) as well as pseudoceramides may also be optionally included as can 10- and/or 12-hydroxystearic acid. Amounts of extract, polyol and these additional materials when used may range from 0.0001 to 3%, and preferably, from 0.001 to 2%, and most preferably, from 0.001 to 1.5% by weight of the end use cosmetic composition. Colorants or dyes may also be included in the disclosed compositions. These substances may range from 0.05 to 5%, preferably, between 0.1 to 2% by weight of the composition.

Conditioning agents like hydroxypropyltrimonium chloride, 5-ureidohydantoin and/or glyoxyldiureide may be used. The components when used make up from 0.5 to 4%, and, preferably, from 0.75 to 4%, and most preferably, from 1 to 3% by weight of the powdered cosmetic composition.

Sunscreen actives may also be optionally included in the disclosed cosmetic powder composition. Particularly preferred are such materials as ethylhexyl p-methoxycinnamate, available as Parsol MCX®, Avobenzene, available as Parsol 1789® and benzophenone-3, also known as Oxybenzone. Inorganic sunscreen actives may be employed such as microfine titanium dioxide, zinc oxide, polyethylene and various other polymers. Amounts of the sunscreen agents when present may generally range from 0.01 to 3%, preferably, from 0.5 to 2%, optimally, from 0.75 to 1.5% by weight of the end use cosmetic composition.

Conventional buffers/pH modifiers may be used in the end use cosmetic composition. These include commonly employed additives like sodium hydroxide, potassium hydroxide, hydrochloric acid, citric acid/citrate buffers, triethanolamine, or mixtures thereof. These materials are added at amounts to obtain the desired pH of the end use cosmetic composition. Upon desired hydration, it is expected that the pH of the powdered cosmetic composition will range from 3.5 to 7.5, preferably, from 4.0 to 6.5, and, most preferably, from 4.5 to 5.5 where pH is determined using a Thermo Fisher Scientific pH meter.

The bulk density of the cosmetic powder composition is typically from 0.2 to 1.2, and, preferably, from 0.3 to 0.9, and, most preferably, from 0.35 to 0.7 g/cm³ where bulk density is measured by art recognized techniques that use, for example, a gas pycnometer made commercially available by Microtrac MRB.

When preparing powdered cosmetic composition, ingredients may be mixed and/or agitated at atmospheric pressure. The temperature at which mixing and agitation occurs is typically from 20° C. to 50° C. Mixing and/or agitation is stopped when a homogeneous mixture is obtained and the resulting powdered cosmetic composition is free of aggregated particles. The particles of the powdered cosmetic composition will have an average particle size from 0.5 to 300 microns, and preferably from 5 to 250 microns, and most preferably, from 10 to 175 microns as determined with art recognized devices such as sieves as noted herein.

Many types of packaging can be used to store and deliver the present cosmetic composition. The selection of packaging is dependent upon the personal care end-use and the viscosity of the composition itself. As an example, leave-on lotions and creams for skin typically employ plastic containers with an opening at a dispense end covered by an appropriate closure. Conventional closures include flip-top hinged lids, screw-caps and non-aerosol pumps. As another example, appropriate packaging to be used for antiperspirants, deodorants and depilatories include a container with a roller-ball applicator on a dispensing end if the composition is fluid and of a thinner viscosity. If the composition is in a stick format, a container with a propel-repel mechanism wherein the stick is fixed on a platform towards a dispensing orifice is appropriate. If the composition is in an aerosol format, then metallic cans pressurized by a propellant and having a spray nozzle is appropriate. In general, patches, bottles, tubes, roller-ball applicators, squeeze containers or lidded jars are preferred.

The cosmetic compositions will now be described below in the context of specific non-limiting examples to facilitate a greater understanding of the present cosmetic compositions. One of ordinary skill in the art will recognize that variations of the inventive compositions that differ from the examples given may be practiced without deviating from the teachings of the present compositions.

### EXAMPLES

All quaternary mixture samples were made by mixing the mentioned ingredients under conditions of moderate sheer, 22° C. to 30° C., and atmospheric pressure, then heating the mixture to 70° C. to 85° C. and atmospheric pressure to result in a molten mixture. The resultant molten mixture may be spray-cooled into the desired particle size, or may be cooled into a solid block which is then milled into the desired particle size to produce a powder. The stabilizing carrier was subsequently added to the quaternary mixture at room temperature (22° C. to 30° C. and atmospheric pressure).

### Example 1: Sample Formulations

Powdered cosmetic compositions were made according to the present invention with the ingredients and amounts set forth in Example 1. The ingredients were combined via agitation at atmospheric pressure and at 25° C. Surprisingly, no visual clumping or agglomeration of composition was observed even after the product was stored at two weeks under representative hot and humid conditions (75% RH at 40° C.). Additionally, the powdered cosmetic compositions had a bulk density from 0.4 to 0.6 g/cm³ and was easily flowable in the absence of visual static cohesion.

| | Out of scope | | | | | | | | | |
|---|---|---|---|---|---|---|---|---|---|---|
| | formulations | | | In-scope formulations | | | | | | |
| Ingredient | Weight Percent (wt. %) | | | | | | | | | |
| Name | A | B | C | D | E | F | G | H | I | J |
| Quaternary | 100 | 85.0 | — | 30.0 | — | — | 40.0 | 40.0 | 40.0 | 40.0 |
| mixture (35% | | | | | | | | | | |
| Behentrimonium | | | | | | | | | | |
| chloride (BTAC); | | | | | | | | | | |
| 55% cetearyl | | | | | | | | | | |
| alcohol; 10% | | | | | | | | | | |
| tapioca starch) | | | | | | | | | | |
| Quaternary | — | — | — | — | 40.0 | 40.0 | — | — | — | — |
| mixture (35% | | | | | | | | | | |
| BTAC; 55% | | | | | | | | | | |
| stearyl alcohol; | | | | | | | | | | |
| 10% tapioca | | | | | | | | | | |
| starch) | | | | | | | | | | |
| BTAC | — | — | 0.8 | — | — | — | — | — | — | — |
| Cetyl Alcohol | — | — | 1.0 | — | — | — | — | — | — | — |
| Stearyl Alcohol | — | — | 1.8 | — | — | — | — | — | — | — |
| Microcrystalline | — | 7.5 | — | 31.8 | 26.8 | — | 26.8 | — | — | — |
| Cellulose | | | | | | | | | | |
| Hydroxypropyl | — | 7.5 | — | Balance | Balance | Balance | Balance | — | — | — |
| Starch Phosphate | | | | | | | | | | |
| Tapioca Starch | — | — | — | — | — | 26.8 | — | — | — | — |
| Bentonite Clay | — | — | — | — | — | — | — | 60.0 | — | — |
| Talc | — | — | — | — | — | — | — | — | 60.0 | — |
| Dimethicone/Vinyl | — | — | — | — | — | — | — | — | — | 60.0 |
| Dimethicone | | | | | | | | | | |
| Crosspolymer | | | | | | | | | | |
| (and) Silica (and) | | | | | | | | | | |
| Butylene Glycol | | | | | | | | | | |
| PEG-200 | — | — | Balance | — | — | — | — | — | — | — |
| Dimethicone | — | — | 4.2 | — | — | — | — | — | — | — |
| 10,000 cSt | | | | | | | | | | |
| Tricalcium citrate | — | — | — | — | — | 5.0 | — | — | — | — |
| Preservative | — | — | — | 0.6 | 0.6 | 0.6 | 0.6 | — | — | — |
| Skin Benefit | — | — | — | 0.03 | 0.03 | 0.03 | 0.03 | — | — | — |
| Agent | | | | | | | | | | |
| Anhydrous | — | — | 15.0 | — | — | — | — | — | — | — |
| Magnesium | | | | | | | | | | |
| Sulfate | | | | | | | | | | |
| Fragrance | — | — | — | 0.6 | 0.6 | 0.6 | 0.6 | — | — | — |
| Stability at 75% | No | No | No | Yes | Yes | Yes | Yes | Yes | Yes | Yes |
| RH and 40° C., 2 | | | | | | | | | | |
| weeks | | | | | | | | | | |

About one (1) gram of powdered cosmetic composition described in the table presented in Example 1 were applied to the left forearm of trained panelists followed by about four (4) grams of water. The panelists, using moderate shear with their right hands were able to mix the water and powdered cosmetic composition to generate an end use cosmetic composition that was not grainy and that provided consumer acceptable conditioning upon use. The flowability of the powder made it easy to remove from packaging with an open orifice and a biodegradable sachet or pouch. Prior to use by the panelists, the powdered cosmetic compositions of Samples O through J, which were made according to the inventive compositions, were assessed and confirmed to be easily hydratable and easily dispensed as free-flowing powders. Sample C was also noted to have been too soft, as the resulting hardness not within the disclosed desired range. Thus, it was not possible for Sample C to be processed into a workable powder.

### Example 2: Impact of Varying Quaternary Mixture Components

| Ingredient | wt. % | | | | | | | | | | | | | |
|---|---|---|---|---|---|---|---|---|---|---|---|---|---|---|
| Name | 1 | 2 | 3 | 4 | 5 | 6 | 7 | 8 | 9 | 10 | 11 | 12 | 13 | 14 |
| Behen- | 24.3 | 35.0 | — | — | — | 35.0 | — | — | 33.0 | 35.0 | 35.0 | 21.0 | 35.0 | 20.0 |
| trimonium | | | | | | | | | | | | | | |
| chloride | | | | | | | | | | | | | | |
| (BTAC) | | | | | | | | | | | | | | |
| Stearyl | — | — | 35.0 | — | — | — | — | — | — | — | — | — | — | — |
| trimethyl | | | | | | | | | | | | | | |
| ammonium | | | | | | | | | | | | | | |
| chloride | | | | | | | | | | | | | | |
| Behen- | — | — | — | 35.0 | — | — | — | — | — | — | — | — | — | — |
| trimonium | | | | | | | | | | | | | | |
| Methosulfate | | | | | | | | | | | | | | |
| Distearyl- | — | — | — | — | 35.0 | — | — | — | — | — | — | — | — | — |
| dimonium | | | | | | | | | | | | | | |
| Chloride | | | | | | | | | | | | | | |
| Cetearyl | 55.7 | 55.0 | 55.0 | 55.0 | 55.0 | — | — | — | 52.0 | 55.0 | 55.0 | 33.0 | 55.0 | 31.0 |
| Alcohol | | | | | | | | | | | | | | |
| Stearyl | — | — | — | — | — | 55.0 | — | — | — | — | — | — | — | — |
| Alcohol | | | | | | | | | | | | | | |
| Glyceryl | — | — | — | — | — | — | 55.0 | — | — | — | — | — | — | — |
| mono- | | | | | | | | | | | | | | |
| stearate | | | | | | | | | | | | | | |
| Steareth-2 | — | — | — | — | — | — | — | 55.0 | — | — | — | — | — | — |
| Pentylene | 5.0 | — | — | — | — | — | — | — | — | — | — | — | — | — |
| Glycol | | | | | | | | | | | | | | |
| Tapioca | — | 10.0 | 10.0 | 10.0 | 10.0 | 10.0 | 10.0 | 10.0 | 15.0 | — | — | 40.0 | — | 8.0 |
| Starch | | | | | | | | | | | | | | |
| Corn Starch | — | — | — | — | — | — | — | — | — | 10.0 | — | — | — | — |
| Rice Starch | — | — | — | — | — | — | — | — | — | — | 10.0 | — | — | — |
| Micro- | — | — | — | — | — | — | — | — | — | — | — | — | 10.0 | 43.0 |
| crystalline | | | | | | | | | | | | | | |
| Cellulose | | | | | | | | | | | | | | |
| Glycerin | 5.0 | — | — | — | — | — | — | — | — | — | — | — | — | — |
| Hydrates | N/A | Yes | Yes | Yes | No | Yes | N/A | N/A | Yes | Yes | Yes | Yes | Yes | N/A |
| instantly | | | | | | | | | | | | | | |
| Hardness | 115 | 316 | 519 | 358 | 724 | 523 | 210 | 68.0 | 348 | 333 | 387 | 388 | 292 | N/A |
| (positive | | | | | | | | | | | | | | |
| force (g)) | | | | | | | | | | | | | | |

It was unexpectedly determined that desirable powdered cosmetic compositions made according to the inventive composition disclosed herein were limited by the components of a quaternary ammonium compound mixture, which dictated whether the resulting powdered cosmetic composition would be processable and/or hydrate instantly. The data demonstrates that each sample hydrated instantly and fell within the desirable range for hardness from 250 to 600 positive force (g). The ingredients of the formulae made in Example 2 were mixed in a manner similar to the one described in Example 1.

The powdered composition of Sample 1 was not made in accordance with the inventive composition and its resulting softness was observed. Samples 2 to 5 illustrate the impact of varying cationic surfactants (i.e., the class of surfactants under which quaternary ammonium compounds belong) in the quaternary mixture. Sample 5 was not made in accordance with the disclosed powdered cosmetic composition. Distearyldimonium chloride (which is commercially available as Varisoft® TA 100 from the supplier Evonik), which is characterized by a double fatty chain attached to a cationic headgroup, was used and did not produce a desirable quaternary mixture, as evidenced by the resulting hardness above the disclosed desired range and inability to be hydrated in water at a desirable speed.

Samples 2 and 6 to 8 demonstrate that, of nonionic surfactants evaluated, fatty alcohols are desirable for use in the inventive compositions disclosed herein, as seen in the optimal hardness achieved in the resulting quaternary ammonium compound mixture. In particular, Samples 2 and 6 demonstrate that fatty acids composed of differing chain lengths may be used in the disclosed powdered cosmetic compositions. Use of cetearyl alcohol, which is a 30:70 ratio blend of cetyl alcohol to stearyl alcohol, in Sample 2 illustrated that a blend of fatty alcohols is also in scope. Use of glyceryl monostearate and steareth-2, however, do not result in desirable quaternary mixtures as the hardness criteria was not met.

Samples 9 to 13 show the effect that varying the identity of hydration enhancer had on the resulting quaternary ammonium compound mixture. Samples 9 to 13 thus illustrated desirable hydration enhancers for use in the inventive compositions. Sample 14 demonstrates that starches in quantities out of scope of the disclosed ranges (e.g., 51% by weight total starch here) resulted in a quaternary mixture that is not processable, wherein a uniform structure was not adequately created. Consequently, hardness and hydration were not able to be measured.

1. A powdered cosmetic composition comprising:
a) a quaternary mixture comprising a quaternary ammonium compound; a C₁₄₋₂₂ fatty acid, fatty alcohol, or combination thereof; and at most 50% a hydration enhancer by weight of the powdered cosmetic composition; and
b) 20 to 90% a stabilizing carrier by weight of the powdered cosmetic composition, wherein the quaternary mixture has a hardness from 250 to 600 positive force (g) and an average particle size from 20 to 250 microns.

2. The powdered cosmetic composition according to claim 1, wherein
the quaternary ammonium compound has the chemical structure:
[R—N(CH₃)₃]⁺X⁻,
wherein R is a C₁₄₋₂₂ alkyl group,
and wherein X is a halogen, phosphate, sulfate, or carbonate anion.

3. The powdered cosmetic composition according to claim 1, wherein the quaternary ammonium compound comprises behentrimonium chloride, stearyl trimethyl ammonium chloride, and behentrimonium methosulfate.

4. The powdered cosmetic composition according to claim 1, wherein the quaternary mixture comprises 10 to 55% quaternary ammonium compound by weight of the quaternary mixture.

5. The powdered cosmetic composition according to claim 1, wherein the C₁₄₋₂₂ fatty acid, fatty alcohol, or combination thereof is characterized by a melting point greater than 40° C. and is present in amounts of 30 to 65% by weight of the quaternary mixture.

6. The powdered cosmetic composition according to claim 1, wherein the quaternary ammonium compound mixture comprises quaternary ammonium compound to fatty acid, fatty alcohol, or combination thereof in a weight ratio of 20:80 to 80:20.

7. The powdered cosmetic composition according to claim 1, wherein the hydration enhancers comprise Zea mays (corn) starch, tapioca starch, Oryza sativa (rice) starch, microcrystalline cellulose, hydroxypropyl starch phosphate, and mixtures thereof in amounts of 2.5 to 40% hydration enhancers by weight of the powdered cosmetic composition.

8. The powdered cosmetic composition according to claim 1, wherein the powdered cosmetic composition comprises 2 to 70% quaternary ammonium compound mixture by weight of the powdered cosmetic composition.

9. The powdered cosmetic composition according to claim 1, wherein the stabilizing carrier comprises Zea mays (corn) starch, tapioca starch, Oryza sativa (rice) starch, microcrystalline cellulose, sodium hydroxypropyl starch phosphate, clay or clay mineral, silicone and/or elastomer powder, or a mixture thereof in amounts of 22 to 80% by weight stabilizing carrier in the powdered cosmetic composition.

10. The powdered cosmetic composition according to claim 1, wherein the composition is a precursor to a hands, face, body, hair, nails, or inanimate object cosmetic composition.

11. The powdered cosmetic composition according to claim 1, wherein the cleansing powder composition is stable and does not agglomerate under conditions of high humidity and high temperature (75% relative humidity at 40° C.) for at least two (2) weeks.

12. The powdered cosmetic composition according to claim 1, wherein the powdered cosmetic composition is in the form of a free-flowing powder easily dispensable through a small orifice.

13. A powdered cosmetic composition comprising the powdered cosmetic composition according to claim 1 hydrated with water, wherein the weight of water used with the powdered cosmetic composition to produce an end use cosmetic composition is from 1 to 12 times the weight of powdered cosmetic composition used.

14. The powdered cosmetic composition according to claim 13, wherein the weight of water used with the powdered cosmetic composition to produce an end use cosmetic composition is from 3 to 10 times the weight of powdered cosmetic composition used.

15. A method for conditioning comprising the steps of:
a) hydrating the powdered cosmetic composition according to claim 1 to produce a powdered cosmetic composition; and
b) contacting skin, hair, nails or an inanimate object with the end use cosmetic composition,
wherein the powdered cosmetic composition is hydrated with shear to produce the cosmetic composition before or after contact with the skin, hair, nails, or an inanimate object.

16. The powdered cosmetic composition according to claim 2, wherein R is a C₁₆₋₂₂ alkyl group.

17. The powdered cosmetic composition according to claim 1, wherein the quaternary mixture comprises 15 to 50% quaternary ammonium compound by weight of the quaternary mixture.

18. The powdered cosmetic composition according to claim 1, wherein the C₁₄₋₂₂ fatty acid, fatty alcohol, or combination thereof is characterized by a melting point greater than 45° C. and is present in amounts of 35 to 60% by weight of the quaternary mixture.

19. The powdered cosmetic composition according to claim 1, wherein the quaternary ammonium compound mixture comprises quaternary ammonium compound to fatty acid, fatty alcohol, or combination thereof in a weight ratio of 25:75 to 75:25.

20. The powdered cosmetic composition according to claim 1, wherein the quaternary ammonium compound mixture comprises quaternary ammonium compound to fatty acid, fatty alcohol, or combination thereof in a weight ratio of 30:70 to 70:30.
